(19) United States
(12) Patent Application Publication
(10) Pub. No.: US 2021/0069351 A1
(43) Pub. Date: Mar. 11, 2021

(54) METHOD FOR SCREENING PAIN INHIBITING SUBSTANCE

(71) Applicant: NEUROVIS INC., Cheonan-si, Chungcheongnam-do (KR)

(72) Inventors: Hyung Gun KIM, Seoul (KR); Guk Hwa JUNG, Cheonan-si, Chungcheongnam-do (KR); Hye Ran PARK, Cheonan-si, Chungcheongnam-do (KR)

(21) Appl. No.: 16/763,194

(22) PCT Filed: Aug. 23, 2018
(86) PCT No.: PCT/KR2018/009735

§ 371 Date: May 11, 2020

(30) Foreign Application Priority Data
Nov. 13, 2017 (KR) 10-2017-0150658
Aug. 22, 2018 (KR) 10-2018-0098078

(51) Int. Cl. A61K 49/00; A61B 10/00

(52) U.S. Cl. CPC A61K 49/0008; A61B 2010/0077; A61B 10/0045

(57) ABSTRACT

The present invention relates to a method for screening a pain-inhibiting substance, said method comprising the steps of: (a) inserting a microdialysis probe into the L1 site of a spinal cord dorsal horn of a neuropathic pain animal model; (b) collecting a first test sample from the L1 site by microdialysis; (c) administering a pain-inhibiting candidate substance into the body of the animal model; (d) after having administered the pain inhibiting candidate substance, then collecting a second test sample from the L1 site by microdialysis; (e) measuring the concentrations of a pain indicator substance in the first test sample and second test sample respectively; and (f) comparing the concentrations of the pain indicator substance in the first test sample and second test sample.

### TECHNICAL FIELD

The present invention relates to a method for screening and evaluating a pain-inhibiting substance.

The present invention was made with financial supporting by the National Research Foundation of Korea (NRF) grant funded by Korea government Ministry of Education (No. 3017R1D1A1B04462).

### BACKGROUND ART

Pain acts as an early warning signal to protect the body from tissue damage, and may be the most important clinical symptom that impairs the quality of life of an organism. However, pain measurement depends on examination of a body temperature, a pulse rate, a respiratory rate (TPR) and a blood pressure, and assessment of the presence and intensity of pain is insufficient.

This is because, in the case of pain assessment, classification methods (Subjective Pain Scoring System, and qualitative evaluation) according to subjective indicators (individual differences, the skill level of an observer, etc.) are well suggested, but an objective pain assessment method has not been established so far.

To assess pain, conventionally, behavioral responses such as mechanical allodynia assessment (von Frey filament test), assessment of a stimulus response by temperature (hot plate, tail flick test), and chemical pain assessment (formalin test) were used. However, conventional assessment methods determine the intensity of pain based on behavioral changes of an animal expected to have pain, or estimate the intensity of pain based on a developed disease. Since behavioral changes caused by a pain response of an animal have to be minutely and sensitively assessed by judgment, these methods were decreased in experimental accuracy and objectivity.

Therefore, there are no objective and effective indicators for the determination and management (administration of medicine such as painkillers and drugs) of patients with various degrees of pain.

Microdialysis is an in vivo sampling technique used to continuously monitor a biochemical phenomenon in living tissue. This technique is based on sampling of endogenous substances from an extracellular space. Spinal cord dorsal horn microdialysis has been technically developed, but still has limitations.

Accordingly, the present inventors have solved problems of behavioral assessment used as a conventional pain assessment indicator and developed a biological indicator which more objectively and quantitatively assesses the intensity of pain, and thus the present invention was completed.

### DISCLOSURE

### Technical Problem

One purpose of the present invention is to provide a method for screening a pain-inhibiting substance.

Another purpose of the present invention is to provide a method for confirming that a pain-inhibiting substance acts on a L1 segment of the spinal cord dorsal horn.

### Technical Solution

To achieve the above object, one aspect of the present invention, there are provides a method for screening a pain-inhibiting substance, which includes the following steps:

(a) inserting a microdialysis probe into a dorsal horn of the spinal cord at the L1 segment in a neuropathic pain (hereinafter, NP) animal model;

(b) collecting a first test sample from the L1 segment by microdialysis;

(c) administering a pain-inhibiting candidate into a body of the animal model;

(d) after the administration of the pain-inhibiting candidate substance, then collecting a second test sample from the L1 segment by microdialysis;

(e) measuring concentrations of pain indicator substance in the first test sample and the second test sample, respectively; and

(f) comparing the concentrations of the pain indicator substance in the first test sample and the second test sample.

The NP model may be a spared nerve injury (hereinafter, SNI) model.

The pain indicator substance may be one or more selected from the group consisting of a neurotransmitter, a neuropeptide and a cytokine, but the present invention is not necessarily limited. Therefore, any of the pain indicator substance known in the art may be used.

The neurotransmitter may be one or more selected from the group consisting of norepinephrine, dopamine, glutamate, γ-aminobutyric acid (GABA) and a dopamine metabolite, but the present invention is not necessarily limited thereto.

The neuropeptide may be substance P or β-endorphin, but the present invention is not necessarily limited thereto.

The microdialysis probe may be inserted into the spinal cord at an angle of 30 to 55 degrees based on the coronal.

In addition, the microdialysis probe may be inserted into the spinal cord to be located on 1.5 to 3.0 mm deep.

The microdialysis probe may be inserted so that the end of the probe faces a cranial direction of the animal model.

When a stimulus is given to a peripheral nociceptor, it is delivered to the spinal cord (transduction), and the stimulus delivered to the spinal cord is then delivered to the cerebral cortex (transmission). In this process, the stimulus is not directly delivered, but passes through a type of inhibitory pathway (modulation). The spinal cord has a descending modulation pathway that controls pain as well as an ascending pathway that delivers pain generated from the periphery to the central nerves. In this process, neurotransmitters interact with cytokines or various peptides, which is involved in controlling of pain sensation.

In the present invention, a neurotransmitter, a neuropeptide and a cytokine in a sample collected from a site where pain sensations converge may be simultaneously analyzed with high sensitivity. Therefore, the present invention may more accurately assess pain because it is more objective than conventional behavioral assessment and collects a test sample from a live animal. In addition, since concentrations of pain-inhibiting candidate may be simultaneously measured, direct pharmacokinetic/pharmacodynamic (PK/PD) assessment is possible.

Neuropathic pain (NP) is generated by damage to the peripheral nervous system and the central nervous system. This occurs by various causes such as trauma, a disease, infection, etc. Like chronic pain, NP is a severe condition that interferes with a patient's mood, quality of life or work efficiency. However, unfortunately, pain therapeutic agent, despite their enormous costs, do not completely relieve patients' pain, but rather cause several side effects. Accordingly, it is necessary to focus on understanding of molecular and cell biological mechanisms of NP.

A spared nerve injury (SNI) model is one of the peripheral nerve injury models, which is created by cutting two types of nerves (the tibial and common peroneal nerves), except one nerve (sural nerve), of three nerve branches. Within 4 days after injury, physical and thermal hyperalgesia occur and persist for approximately several weeks to 6 months. This model has a relatively easy surgical procedure, and a smaller error in the extent of pain expression, compared to previous models.

Step (a) of the present invention is for inserting a microdialysis probe into a L1 segment of dorsal horn in spinal cord of a NP animal model.

The NP animal model may be a rodent, preferably, a rat, and more preferably, a 180 to 200 g SD-rat. The SD-rat may be manufactured by inhalation anesthesia with 2% isoflurane, fixing the left paw with a tape on the station of a surgical microscope to straighten the leg, incising the back of the knee to widen a space between muscles, making knots with sutures at both ends of the common peroneal nerve and the tibial nerve from the connective tissue, and making a cut between the common peroneal nerve and the tibial nerve and then closing the incision. Before making a cut between the common peroneal nerve and the tibial nerve, it is preferable that the common peroneal nerve and the tibial nerve are separated. In addition, the suture may be 7-0 suture, and the spacing of knots may be 0.5 to 1.5 cm, and preferably 1 cm.

Step (b) is for collecting a first test sample by microdialysis.

Step (c) is for administering a pain-inhibiting candidate substance into the animal model.

The pain-inhibiting candidate substance may be administered into an animal model by various methods, preferably intraperitoneal injection.

Step (d) is for collecting a second test sample by microdialysis.

Unlike a common method of measuring a concentration by disrupting tissue to measure a neurotransmitter, since a test sample is directly collected and analyzed from an anesthetized animal model in the present invention, the denaturation of a substance to be measured may be minimized, and the change in concentrations of a neurotransmitter and a candidate substance according to the administration of the candidate substance may be measured in the same animal model, and therefore, the present invention may more accurately and objectively screen a pain-inhibiting substance than a conventional analysis method.

Step (e) is for measuring concentrations of a pain indicator substance in the first test sample and the second test sample, respectively.

According to an embodiment of the present invention, the pain indicator substance may be one or more selected from the group consisting of glutamate, GABA, a neurotransmitter, a neuropeptide and a cytokine.

A substance to be measured can be measured to generally assess pain, and a substance that decreases or increases a concentration of the substance may be screened as a pain-inhibiting substance.

Meanwhile, a ratio of a glutamate concentration to a GABA concentration was calculated, and then a candidate substance that reduces the ratio after administration may be selected as a pain-inhibiting substance.

Gamma-aminobutyric acid (GABA) is an inhibitory neurotransmitter, and acts on the central nervous system of a mammal. When a nerve is excited, GABA serves to control nerve excitation. Excitation is caused by glutamate release, and glutamate is an excitatory neurotransmitter. These two substances are formed and reabsorbed in nerve cells, and maintain homeostasis in the nervous system. The surface of the dorsal horn is the first place that receives a pain signal in the central nervous system and where a primary afferent nerve delivers a stimulus to the brain through an ascending pathway.

According to an embodiment of the present invention, the neurotransmitter may be one or more selected from the group consisting of norepinephrine, dopamine, glutamate, GABA and a dopamine metabolite.

According to an embodiment of the present invention, the neuropeptide may be substance P or β-endorphin.

According to an embodiment of the present invention, the cytokine may be one or more selected from the group consisting of MIP-1α, C5α, TNF-α, IL-1β, IL-6, IL-15, IL-18, IFN-γ, MCP-1, CXCL1, EAA, PGEs, ATP, Nitric oxide, BDNF, c-Fos and LTs.

The final step (f) of the present invention is for comparing the concentration of the pain indicator substance of the first test sample with the concentration of the pain indicator substance of the second test sample.

While concentrations of the substances may be measured by a common method known in the art to measure a concentration of a target substance included in the test samples, various target substances are preferably analyzed simultaneously using a mass spectrometer.

Another aspect of the present invention provides a method for screening a pain-inhibiting substance, which includes the following steps:

(a) inserting a microdialysis probe into a L1 segment of a dorsal horn in spinal cord of a neuropathic pain animal model;

(b) collecting a first test sample from the L1 segment by microdialysis;

(c) administering a pain-inhibiting candidate substance into a body of the animal model;

(d) after the administration of the pain-inhibiting candidate substance, collecting a second test sample from the L1 segment by microdialysis;

(e) measuring ratios (Glu/GABA) of concentration of a glutamate to the concentration of a γ-aminobutyric acid (GABA) concentration in the first test sample and the second test sample, respectively; and

(f) comparing the ratios of Glu/GABA concentration in the first test sample and the second test sample.

Compared to the first test sample, when the Glu/GABA ratio of the second sample is decreased, the pain-inhibiting candidate substance may be selected as a pain-inhibiting substance.

A step corresponding to the above-described step in the method of screening a pain-inhibiting substance may have the same meaning as described above.

In the present invention, it was confirmed that pain may not be exactly assessed only with the change in concentration of GABA or glutamate, and the ratios of the glutamate concentration to the GABA concentration is an indicator that can be used to assess pain. Specifically, it was confirmed that when pain is suppressed, the ratios decreases, and therefore, the ratios are measured in test samples collected before and after the administration of a pain-inhibiting candidate substance, and a substance that reduces the ratio may be selected.

Still another aspect of the present invention provides a method for confirming that the pain-inhibiting substance acts on a L1 segment of the spinal cord dorsal horn, which includes the following steps:

(i) inserting a microdialysis probe into a L1 segment of a dorsal horn in spinal cord a NP animal model;

(ii) collecting a first test sample from the L1 segment by microdialysis;

(iii) administering a pain-inhibiting substance into a body of the animal model;

(iv) after the administration of the pain-inhibiting substance, collecting a second test sample from the L1 segment by microdialysis for a predetermined time;

(v) measuring concentrations of a pain indicator substance in the first test sample, and concentrations of a pain indicator substance and a pain-inhibiting substance in the second test sample; and

(vi) confirming the change in concentrations of pain indicator substance in the first test sample and the second test sample, and the change in concentration of a pain-inhibiting substance in the second test sample.

According to an embodiment of the present invention, the microdialysis probe may be inserted into the spinal cord at an angle of 30 to 55 degrees, and preferably, 35 to 45 degrees, based on the coronal.

According to an embodiment of the present invention, the microdialysis probe may be inserted into the spinal cord to be located on 1.0 to 3.0 mm deep, and preferably 2.5 mm deep.

According to an embodiment of the present invention, the microdialysis probe may be inserted so that the end of the probe faces a cranial direction of the animal model.

According to an embodiment of the present invention, the microdialysis probe may be located on a dorsal horn of the spinal cord.

The term “dorsal horn” used herein is a region of the spinal cord which serves as a path of dorsal root ganglia, and accounts for a very small part of the spinal cord. Therefore, to collect a test sample from the dorsal horn, the direction, angle and insertion depth of a microdialysis probe have to be precisely adjusted.

According to an embodiment of the present invention, the microdialysis probe may have a molecular cutoff of at least 30,000 to 80,000 daltons.

Generally, the molecular cutoff of a microdialysis probe such as CMA 7, CMA 10, CMA 11 or CMA 12, which is used to analyze a neurotransmitter, is 6,000 or 20,000 daltons, and the molecular weights of many cytokines are 30,000 to 80,000 daltons. In the present invention, neurotransmitters and cytokines were simultaneously analyzed by using CMA 8 and/or CMA 12 microdialysis probe(s) having a molecular cutoff of 30,000 to 80,000 daltons or more, and preferably 100,000 daltons or more.

The term “pharmacokinetics (PK)” used herein refers to the study of a quantitative time course of drug absorption, distribution, metabolism and removal.

The term “pharmacodynamics (PD)” used herein refers to the study of biochemical and physiological effects and mechanisms of drugs, and includes the study of the interaction between the chemical structure of a drug and the action and effect thereof, and the action and effect of a specific drug.

In the present invention, a simultaneous analysis of concentrations of an intraperitoneally administered drug in a microdialysate test sample collected from an animal model, and simultaneous measurement of a concentration of a drug actually acting on target tissue through the brain-blood barrier were successful. That is, the method for screening a pain-inhibiting substance of the present invention may be used in simultaneous evaluation of actual pharmacokinetics and pharmacodynamics (PK/PD) for a drug acting on the central nervous system.

### Advantageous Effects

Since methods for screening and evaluating a pain-inhibiting substance can simultaneously analyze a neurotransmitter, a neuropeptide and a cytokine in neuropathic pain (NP) animal models, the methods can be effectively used in development and evaluation of a substance effective in pain inhibition and a biomarker.

### DESCRIPTION OF DRAWINGS

FIG. 1 shows images illustrating a process of manufacturing a neuropathic pain (NP) rat model. Using a surgical knife, the left hindlimb skin of the rat was incised to expose gastrocnemius (A). Subsequently, a nerve (sural nerve) to remain was exposed, and a biceps femoris muscle was widened using a retractor (B). A tibial nerve was cut with scissors at mid-spot between each end at 5 mm intervals which tied with silk suture (C, D). And the same method was used to cut the common peroneal nerve (E, F).

FIG. 2 shows images illustrating a von Frey filament test: (A) von Frey filaments, (B) stimulating rat left hindpaw using a von Frey filament, (C) A table of behaviour patterns and threshold, (D) Area of stimulation on the rat the left hindpaw.

FIG. 3 shows images of a lumbar laminectomy surgery of rat and microdialysis of spinal cord dorsal horn. Ear bars held between L2 and L3 vertebral level to fix the vertebra because of minimizing the bone movement by breathing, L1, 2, 3 vertebrae of the rat were exposed (A). The spinal cord of L2 and a part of L3 were exposed after grind with micro hand piece and eliminated bone with micro rongeur (B). And we slightly inserted CMA 7 guide cannula into the spinal cord about angle of 45 degrees from horizontal (C). For conducting in vivo microdialysis, artificial cerebrospinal fluid (aCSF) was perfused through the dorsal horn of spinal cord which inserted CMA 7 probe (D).

FIG. 4 shows diagrams about insertion of a guide cannula and a microdialysis probe into a rat spinal cord: The overall drawing shown for the spinal cord microdialysis of the anesthetized rat and guide cannula which held to stereotaxic holder was inserted into the spinal cord (A). And the microdialysis probe actually inserted at about angle of 45 degrees from horizontal at L1 and a part of L2 of the spinal cord (B).

FIG. 5 shows thresholds of mechanical stimulation in neuropathic pain model and normal rat. Qualification difference of pain sensitivity was expressed a graph of vertical scatter plot. The threshold was significantly low in the NP animal model (***p<0.0001).

FIG. 6 shows a set of calibration curves for γ-aminobutyric acid (GABA) and glutamate analyzed in microdialysates containing actual GABA and glutamate concentrations, respectively. Data were shown the calibration curves for GABA (A) and glutamate (B). The recovery rate of the probe was represented by a correlation graph of the concentration of the analyte relative to the actual concentration using CMA 7 microdialysis probe by in vitro microdialysis. All graphs had a significant correlation (***p<0.0001).

FIG. 7 shows sections stained with H&E(hematoxylin and eosin) staining to identify of inserted probe tip location into the dorsal horn of spinal cord. SNI(spared nerve injury) rat model (A, B) and normal rat (C, D) were shown. If the probe was correctly inserted into the left dorsal horn, it would looks like A and C, otherwise it would looks like B and D (×100, Magnified).

FIG. 8 shows chromatograms for GABA and glutamate: Left for GABA, Right for glutamate, LLOQ(lower limit of quantification) (A; 1 ng/mL for GABA, B; 5 ng/mL for glutamate), HLOQ(higher limit of quantification) (1000 ng/mL) (C, D), and real sample (E, F) in the rat spinal microdialysates.

FIG. 9 shows Concentrations of GABA and glutamate in the SNI model and the control. The concentration of GABA was significantly lower in the SNI group than the control group (A). The concentration of glutamate showed no significant difference between the SNI group and the control group (B). However, the glutamate/GABA ratio was found in the same test sample was significantly higher in the SNI model group compared to the control group (C, *p<0.05).

FIG. 10 shows a correlation of GABA, glutamate concentration and glutamate/GABA ratio compared to the threshold of mechanical stimulation. Graphs showed using linear regression and scattered dots. Dots represented the concentration of each analytes in the SNI model group. Same color of the dot means same individual. Linear regression was represented the correlation between the concentrations of analytes and thresholds of von Frey filament test (***p<0.001).

FIG. 11 shows time variation of the concentration of neurotransmitters in the spinal cord of normal rat group.

FIG. 12 shows graphs of results of measuring concentrations of β-endorphin, substance P and MIP-1α in a NP animal model and a control model by time zone, respectively.

FIG. 13 shows various neuropeptides and cytokines associated with bone metabolism, cardiovascular metabolism, cytokines, inflammation and neuroscience, which may be confirmed in the present invention.

FIG. 14 shows various neuropeptides and cytokines associated with metabolism, endocrinology and toxicity, which may be confirmed in the present invention.

FIG. 15 shows graph of results of measuring concentration of GABA in microdialysate test samples of NP animal model and control model by time zone.

FIG. 16 shows graph of results of measuring concentration of glutamate in microdialysate test samples of NP animal model and control model by time zone.

FIG. 17 shows graph of results of measuring concentration of glutamate/γ-aminobutyric acid (glu/GABA) in microdialysate test samples of NP animal model and control model by time zone.

FIG. 18 shows graph of results of measuring concentration of pregabalin in microdialysate test samples of NP animal model and control model by time zone.

### MODES OF THE INVENTION

Hereinafter, the present invention will be described in further detail with reference to examples thereof. These examples are merely provided to more fully describe the following examples are given for illustration of the present invention only, and are not intended to limit the scope of the present invention, as will be apparent to those skilled in the art.

### EXAMPLE 1

### Method

1-1: Animal

Sprague-Dawley (SD) male rats (Koatech, South Korea) weighing 100˜150 g, were used to generate neuropathic pain rat models. The rats were maintained under specifically controlled conditions (ambient temperature 23±2° C., 12-h light/dark cycle).

All procedures complied with Institutional Animal Care and Use Committee of Dankook University (IACUC, South Korea), which adheres to the guidelines issued by the Institution of Laboratory of Animal Resources.

1-2: Neuropathic Pain Rat Model

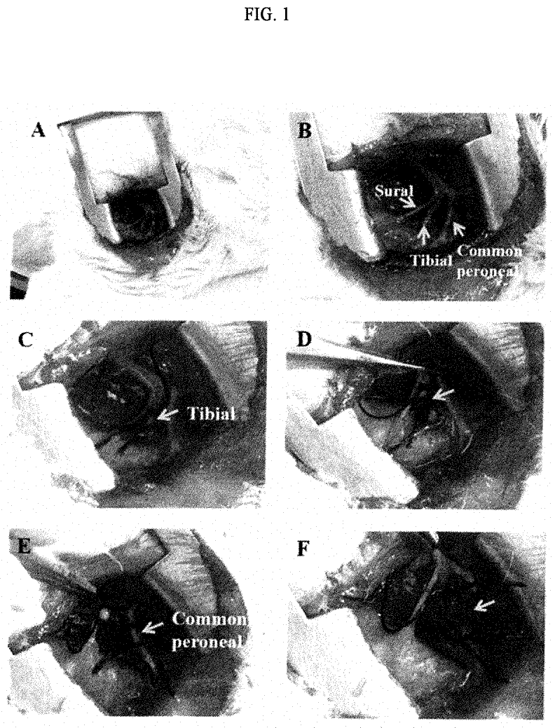

Spared nerve injury (SNI) models were used to assess the concentration of a pain indicator substance present in a dorsal horn in the spinal cord. To generate an efficient neuropathic pain model, the rats were anesthetized under 3% isoflurane (Hana Pharm., South Korea), after incision on the left hind of the rat leg the common peroneal and the tibial nerve of three peripheral nerve branches in the sciatic nerve were axotomized and the sural nerve was spared then the surgical site was closed (FIG. 1). After surgery, animals were placed in the home cage to recover. 2 weeks after surgery, hypersensitivity will occur in the lateral area of the rat left hind paw.

1-3: Von Frey Filament Test

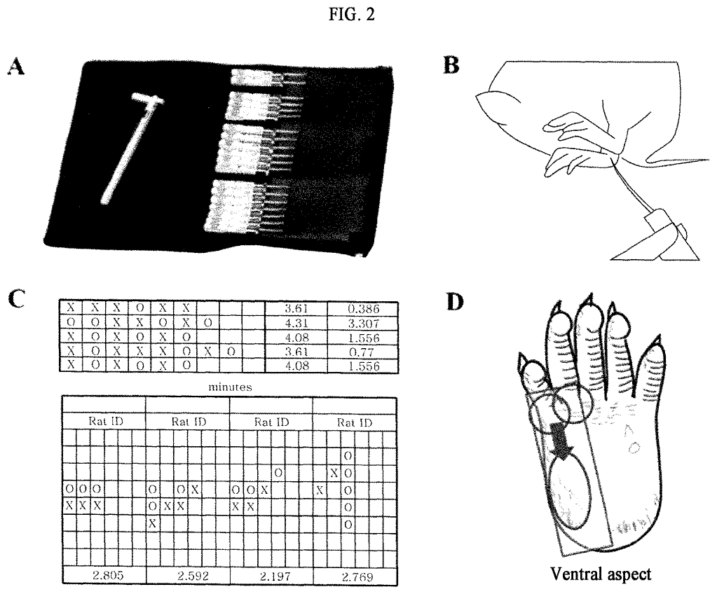

2 weeks post-surgery, we applied von Frey filament test established by 50% up and down threshold method for evaluate mechanical allodynia in SNI model (Chaplan, S. R., et al., 1994. Quantitative assessment of tactile allodynia in the rat paw. Journal of Neuroscience Methods, 53(1), 55-63.). The rats were habituated 5 minutes in the apparatus. For determining which animal is sensitive to the stimulus, we used 0.4 g, 0.6 g, 1 g, 2 g, 4 g and 6 g of von Frey filament (Stoelting, USA), and excluded animals from the experiment who did not respond to the stimulus of the filament. We stimulated 5 times in aspect of rat left paw using each filament from thick to thin (FIG. 2). If the rat response to stimulate over 3 times, we considered that the rat has NP. The pain response was determined by the behaviours of the rats suddenly taking off their foot and shrank or licking their foot with their tongue. The behavioural patterns of the pain were recorded to calculate the threshold value of the pain. For example, if the rats were responding from 2 g to 0.4 g filaments sequentially, the pattern of threshold is 0.4 g, and if the rats were not responding from 2 g to 15 g filaments sequentially, the threshold is 15 g. The other behavioural patterns were calculated according to the formula below.50% g threshold=(10[Xf+kδ])/10,000

(Xf; The value of the von Frey hair used for the last measurement, k; The tabular value of the Positive/Negative response pattern, δ; Mean difference (in log units) of stimulus. In this formula, 0.224 was applied en bloc.)

1-4: Lumbar Laminectomy Surgery

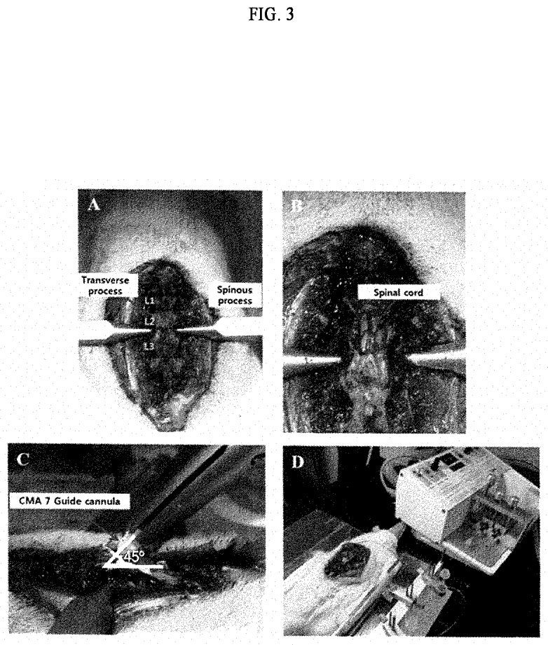

Rats were anesthetize with isoflurane, starting at 3%, and maintained at 1%. The anesthetize rats were mounted on a stereotaxic instrument (David Kopf instruments, USA) and surgically exposed by incising the back muscle by incising between the ligament and the back bone. After eliminating the muscle of vertebrae, lumbar 1 (L1) and lumbar 2 (L2) vertebrae were also eliminated for exposing spinal cord by micro rongeur (Fine Science Tools, USA) and micro motor hand piece (SAESHIN, South Korea). A surface of the lumbar L2 of vertebra was exposed and held transversal process between L2 and L3 vertebral level on the horizontal plane with the ear bars of stereotaxic instrument for rat (David Kopf instruments, USA) (FIG. 3). The durameter of L2 spinal cord were opened carefully and a microdialysis guide cannula (CMA 7 Guide cannula, CMA Microdialysis AB, Sweden) was slightly inserted into the L2 spinal cord. Inserted guide cannula was tilted about angle of 45 degrees from horizontal. To fix the guide cannula to the backbone, dental cement (Dentsply Sirona, USA) was applied between the bone and the back of guide cannula tilted from the backbone.

1-5: In Vitro Microdialysis

We conducted in vitro microdialysis to determine the neurotransmitter concentration in the tissue that we inserted into the spinal cord tissue, and to find out concentration of substance in a test sample through the membrane of microdialysis probe. We used CMA 7 microdialysis probe (CMA Microdialysis AB, Sweden) and working solutions of actual concentration of GABA and glutamate dissolved in aCSF composed with 147 mmol/L of NaCl, 2.7 mmol/L of KCl, 1.2 mmol/L of CaCl2, and 0.85 mmol/L of MgCl2 (Artificial cerebrospinal fluid, CMA Microdialysis AB, Sweden) to conduct quality control and to determine recovery of microdialysis probe. Working solutions were prepared 10 (LLOQ, Lower limit of quantification), 50 (LQC, Low quality control), 1000, 5000 (MQC, Middle quality control), 8000 ng/ml (HQC, High quality control) of GABA (A2129, Sigma Aldrich, USA) and 50 (LLOQ), 100 (LQC), 1000, 5000 (MQC), 8000 ng/ml (HQC) of glutamate (49449, Sigma Aldrich, USA) in micro-centrifuge tube to analysis using LC-MS/MS. The microdialysis probe was soaked in working solutions and perfused with aCSF (artificial cerebrospinal fluid). A flow rate was 1.0 ul/min and time interval were 30 minutes on the same condition as in vivo microdialysis.

1-6: In Vivo Microdialysis

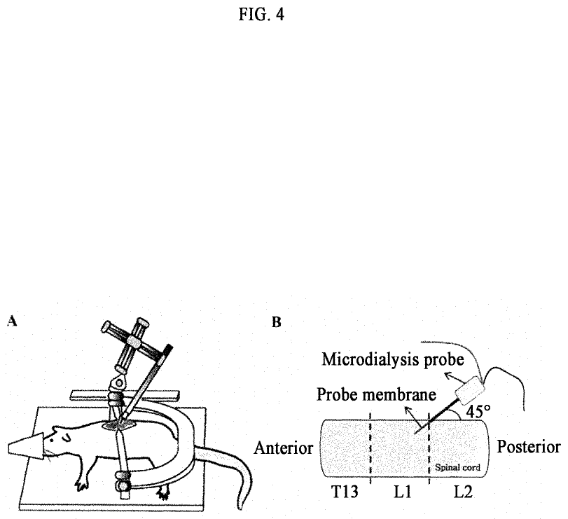

Before inserting microdialysis probe into the tissue, the probe was immersed in 70% ethanol and washed for 15 minutes at the flow rate of 3.5 ul/min. Then, the probe was immersed in distilled water and washed for 15 minutes in the same protocol. When the washing step was completed, the probe was inserted at a depth of approximately 2 mm into the dorsal horn of the L1 spinal cord (FIG. 4). Before to spinal cord microdialysis sampling, to stabilize a nerve response to mechanical stimulus by the insertion of the microdialysis probe, samples were not collected for approximately 1 hour. However, the probe was perfused on the L1 of the spinal cord continuously with aCSF (artificial cerebrospinal fluid) while stabilization. During the sampling process (FIG. 3), perfusate was also used aCSF, we collected samples 2 times, every 30 minutes, and flow rate was 1 ul/min.

Herein, the microdialysis probe was inserted into the spinal cord of the L1 vertebra at an angle of 45 degrees based on the coronal to be located on 2.5 mm deep.

Following the completion of baseline test sample collection, pregabalin, which is a drug known to have an effect on neuropathic pain, was intraperitoneally injected with pregabalin of 10 mg/kg, and test samples were collected again every 1 hour for 6 hours.

1-7: Storage and Pretreatment of Test Samples

The test samples collected in the <1-4> and the <1-5> were stored in a freezer at −70° C., and thawed at room temperature before use. And, 20 μl of acetonitrile was added to 20 μl of the collected microdialysate and well mixed (vortexing, 30 sec) and resulting mixture was centrifuged at 3000 rpm for 5 minutes to recover a supernatant, thereby performing pretreatment of the test samples.

1-8: LC-MS/MS Analysis

The test samples were analyzed by Agilent HP 1290 series HPLC (Agilent Technologies, Palo Alto, Calif., USA) and triple quadrupole tandem mass spectrometry (API 4000, Applied Biosystems, USA). HPLC columns used were Luna C8 (Phenomenex, USA), 10 mm length×2.0 mm id×3.0 μm particle size.

Preparation of Standard Solution

GABA and glutamate were dissolved acetonitrile to make 1 mg/mL of stock solutions and the stock solution serially diluted with acetonitrile to have concentrations of 1,000, 500, 200, 100, 50, 20 and 10 ng/mL.

Storage and Pretreatment of Test Samples

The test samples were stored in a freezer at −70° C., and thawed at room temperature before use. 20 μL of microdialysate was added to 20 μL of acetonitrile, well mixed (vortexing, 30 sec) and centrifuged at 3,000 rpm for 5 minutes. A supernatant was taken and injected by 5 μL for LC-MS/MS analysis.

Conditions for Device Analysis

The test samples after the pretreatment were analyzed using LC-MS/MS under the conditions shown in Table 1.Mobile phase was used aqueous solution including 0.1% formic acid and acetonitrile, and analysis was performed under isocratic elution conditions at a flow rate of 0.3 ml/min. An oven temperature of an analysis column was constantly 40° C.

Electrospray ionization (ESI) method was selected for MS/MS analysis conditions for detection, and in a positive ionization mode, a multiple reaction monitoring (MRM) method was used. A nitrogen gas was used as a spray gas, a temperature was 500° C., and an ion spray voltage was 4,500 V. Other MS/MS analysis parameters were set to have an entrance potential of 10 V, collision energy of 10 V and a collision cell exit potential of 12 V for analysis.

TABLE 1
LC conditions

| LC-MS/MS system | AB Sciex 4000 coupled with | |
|---|---|---|
| | Agilent 1290 HPLC system | |
| Analytical column | Luna C8 (100*2.0 mm, | |
| | 3 μm, Phenomenex, USA) | |
| Mobile phase | 5% B (A: 0.1% formic acid | |
| | in water, B: acetonitrile) | |
| Flow rate | 0.35 | mL/min |
| Column oven temperature | 45° | C. |
| Injection volume | 5 | μL |
| Run time | 2.5 | min |

MS/MS conditions

| Polarity | Positive | |
|---|---|---|
| Turbo gas | Nitrogen | |
| Curtain gas (CUR) | 10 | psi |
| Turbo gas pressure | 60 | psi |
| Source temperature | 500° | C. |
| Ion spray voltage | 4500 | V |
| Entrance potential (EP) | 10 | |
| Collision energy (CE) | 10 | V |
| Collision cell exit | 12 | V |
| potential (CXP) | | |

1-9: Cytokine Analysis in Microdialysate

For analysis of the neuropeptide and cytokine of the test samples was used using “Luminex 200 multiplex system” by multiplex cytokines assay. Ten most important of pain-related neuropeptides such as IFN-gamma, IL-1 beta, IL-6, IL-10, TNF-α, MCP-1, MIP-1α, BDNF, substance P and β-endorphin and cytokines were analyzed using a total of 4 kits.

1-10: Simultaneous Analysis of Drugs

Pregabalin is administered intraperitoneally at a concentration of 30 mg/kg in the neuropathic pain model and the control rats manufactured in the Example <1-2>, and the concentration of pregabalin were observed for 6 hours under the same conditions as the analysis of the neurotransmitter to simultaneously measure the concentrations of drugs acting on a dorsal horn of the spinal cord, which is a drug action point.

1-11: Confirmation of Location of Microdialysis Probe

After the collection of the test samples in the Example <1-4> and only the spinal cord was isolated, to confirm whether the microdialysis probe was correctly inserted into dorsal horn tissue of the spinal cord to complete the surgery, histological staining and examination were performed on the cutting plane and side of the spinal cord, and the insertion location of the microdialysis probe was confirmed.

1-12: Hematoxylin & Eosin Staining (H&E Staining)

The tissue of spinal cord stored at 10% formalin solution was dissected 2-3 mm of thickness using micro blade. Then, the tissue was fixed in paraffinized for 13 hours. The slices of tissue were attached to the slide glass to dry, and the paraffin was removed and washed with distilled water. Hematoxylin and eosin staining was conducted about 10 minutes and 2 minutes each. The dyed slices of tissue were identified through an optical microscope (Axio Scope Al, Zeiss, Germany) where the probe tip was inserted. If a position of the probe at the dorsal horn of the spinal cord was seemed incorrect, we excluded the analysis data.

1-13: Statistical Analysis

Data was presented as means±SEM. All statistical analysis performed using GraphPad Prism 5 software (GraphPad, USA) followed by unpaired t-test for qualitative and quantitative comparisons of neurotransmitters between neuropathic pain rats and control rats. Linear regression analysis assay was used for the comparing between neurotransmitter concentration and behavioural data from each group and used for confirming recovery yield of microdialysis probe. P-values <0.0001, <0.01 and <0.05 were considered to be statistically significant.

### EXAMPLE 2

### Pain Scoring of Mechanical Allodynia

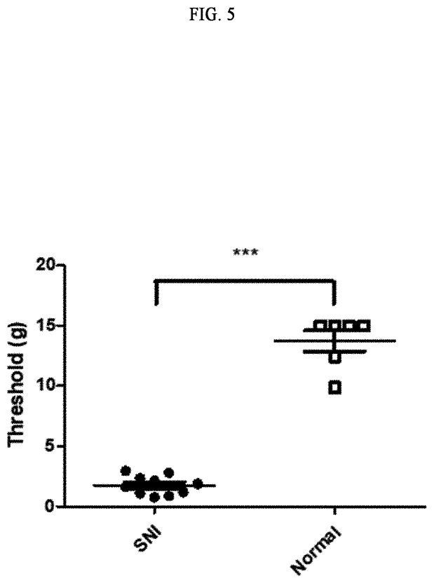

2 weeks after surgery, when a mechanical stimuli were applied to the left paw of rats using von Frey filaments, thresholds were calculated by looking at the behavioural patterns. The thresholds were assessed to be sensitive to pain were less than about 2 g (1.781±0.2517 g) differ from normal rats that were over 9 g (13.72±0.8755 g) of the threshold (FIG. 5).Thus, in terms of behavioural patterns, it was found that sensitivity of neuropathic pain(NP) model was approximately 13 times greater than that of the control. Through this test, the behavioural patterns appeared when mechanical stimuli were applied to the neuropathic pain model and expressed as threshold which was a qualitative assessment of the pain scoring.

### EXAMPLE 3

### Recovery Rate of Microdialysis Probe

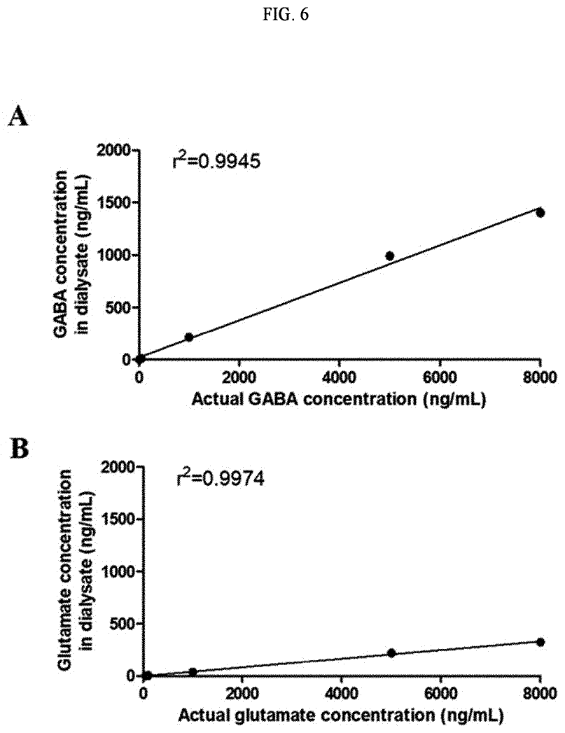

In order to determine the total concentration of neurotransmitter present in the tissue, the recovery rate of the microdialysis probe must be known. As some of endogenous substance in the extracellular fluid released through semipermeable membrane are present in the microdialysis probe. The concentration of a dialysate produced through the CMA 7 microdialysis probe was evaluated using LC-MS/MS. As a result, GABA and glutamate in the microdialysate had a high correlation with actual concentrations and produced a linear calibration curve (GABA; r²=0.9945, glutamate; r²=0.9974) (FIG. 6). When the experiment was conducted at the same flow rate and condition as in vivo microdialysis, the recovery rate of GABA was approximately 20% from actual concentration, and the recovery rate of glutamate was around 4.8% (Table 2). A slope of the calibration curve of the GABA recovery rate (0.1784±0.0078) was higher than that of the glutamate recovery rate (0.0418±0.0012), indicating that the GABA recovery rate in the microdialysis probe is relatively higher than the glutamate recovery rate.

TABLE 2

| GABA | | glutamate | |
|---|---|---|---|
| Actual | | Actual | |
| concentration | microdialysate | concentration | microdialysate |
| (ng/mL) | (ng/mL) | (ng/mL) | (ng/mL) |
| 10 | 2.0 | 50 | 1.0 |
| 50 | 15.0 | 100 | 3.9 |
| 1,000 | 189.0 | 1000 | 50.0 |
| 5,000 | 900.0 | 5000 | 199.0 |
| 8,000 | 1,480.0 | 8000 | 317.0 |

### EXAMPLE 4

### Confirmation of Location of Probe Tip Into the Spinal Cord Dorsal Horn

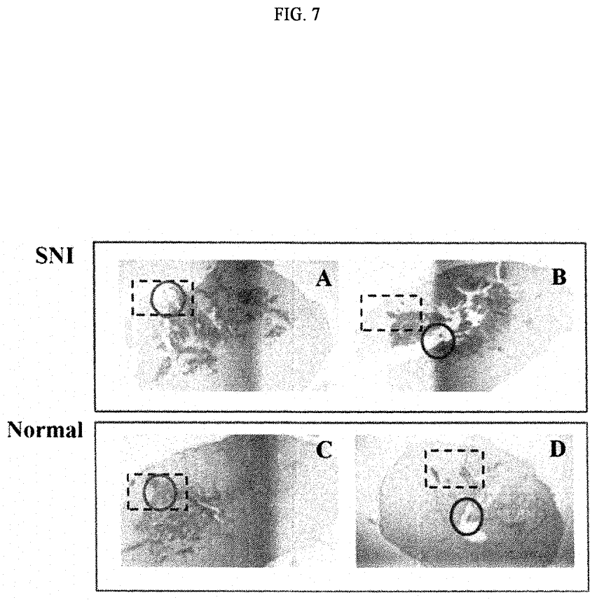

To confirm the microdialysis probe was exactly inserted in the dorsal horn of L1 spinal cord, an insertion trace was confirmed using H&E staining. Since an axotomized sciatic nerves were connected to L1 of spinal cord, correctly targeting had to be confirmed in order to accurately assess the pain. After checking a dyed tissue slices of spinal cord, we found the trace in all controls and SNI models (FIG. 7).

### EXAMPLE 5

### Quantification of GABA and Glutamate in Microdialysate in Spinal Cord

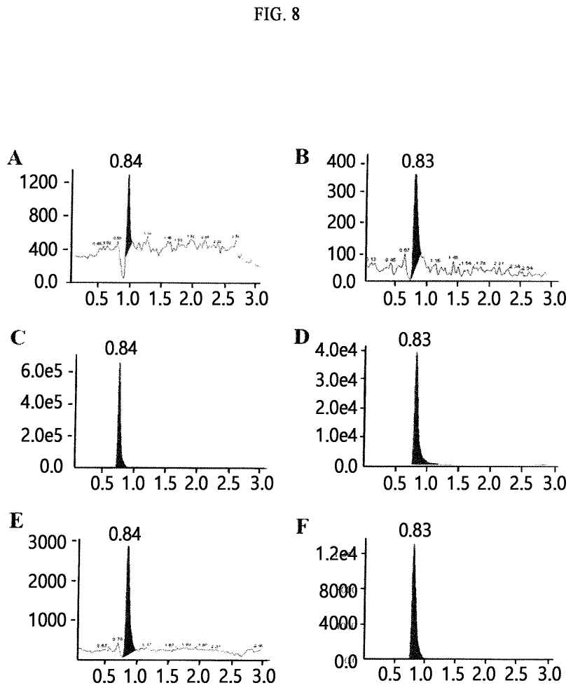

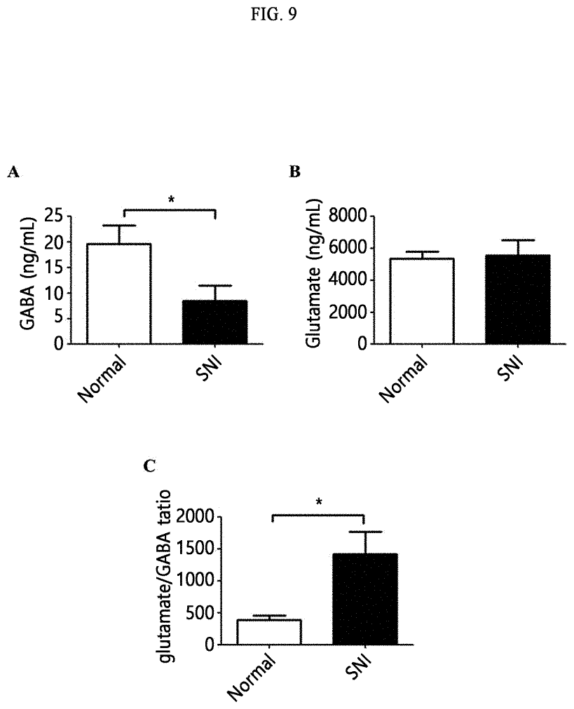

Using LC-MS/MS, the amount of GABA and glutamate in the micro dialysates of the spinal cord was quantified with representative chromatograms (FIG. 8). LLOQ, HQC and the real analysis value were all highly responsible on the detector and the peaks were well formed. Quantification of GABA and glutamate was carried out using MRM(Multiple reaction monitoring) of m/z 104.0→87.0, and 148.0→84.0, respectively. The quantitative data obtained by averaging the concentration of 2 dialysate samples collected every 30 minutes and then multiplied by the recovery rate of the microdialysis probe to calculate the amount of GABA and glutamate present in the tissue using an unpaired t-test. In the L1 spinal cord of SNI model, it was found that GABA was about twice smaller (9.082±3.257 ng/mL) (FIG. 9) than the control group (19.59±3.593 ng/mL), and both groups had similar amounts of glutamate (SNI; 5527.0±999.3 ng/mL, Normal; 5328.0±474.6 ng/mL). Calculating the ratio of two neurotransmitters, the amount of glutamate to GABA in the SNI model was approximately 3.6 times higher with a significant difference (SNI; 1412.0±352.6 ng/mL, Normal; 390.4±69.52 ng/mL). That allowed us to assess the presence of pain through the difference in the amount of GABA in the SNI model, one of the neuropathic pain models.

### EXAMPLE 6

### Correlation of GABA, Glutamate Concentration and Threshold of Mechanical Allodynia in the Neuropathic Pain Model

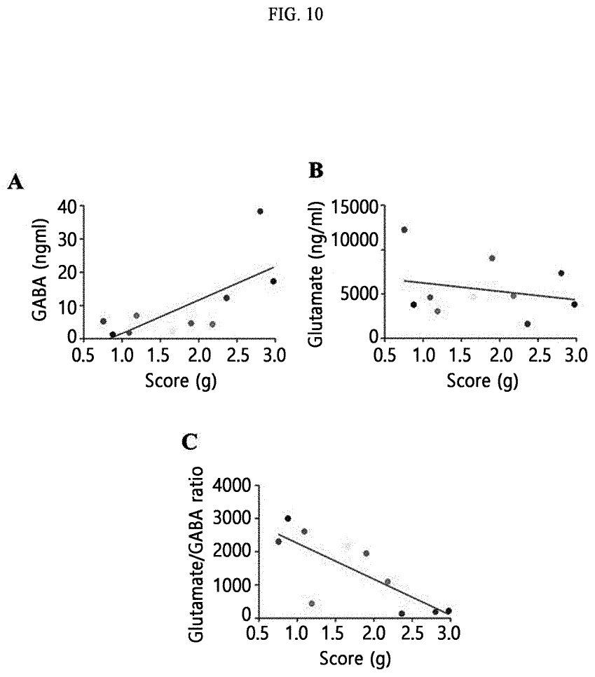

In previous studies, pain was mostly assessed by applying mechanical stimulation and scoring patterns of animal behaviour. Depending on the severity of pain, we compared the concentration of neurotransmitters in the living spinal cord tissue and evaluated it, measured on the SNI model and statistics were used linear regression. As a result, the lower the pain threshold value of the SNI model, the lower amount of GABA in the spinal cord, showed a proportional curve (y=10.04×8.412) (FIG. 10), which had a significant correlation (p=0.0224). On the other hand, glutamate in the spinal cord showed a pattern that was not significantly related to the threshold of pain (p=0.5728). Taken together, the lower the glutamate/GABA ratio in the spinal cord of the SNI model, the higher inverse value of the pain (y=−1082×+3339), that had highly significant correlation (p=0.0089). This means that the greater the pain in the SNI model, the higher the glutamate/GABA ratio, as the amount of GABA decreased without any quantitative changes in glutamate. Compared with the previous results (FIG. 9) the means of concentration of GABA, glutamate and glutamate/GABA ratio in the SNI model group and the control group, also showed quantitative patterns of similar correlation with pain threshold in the SNI model individually.

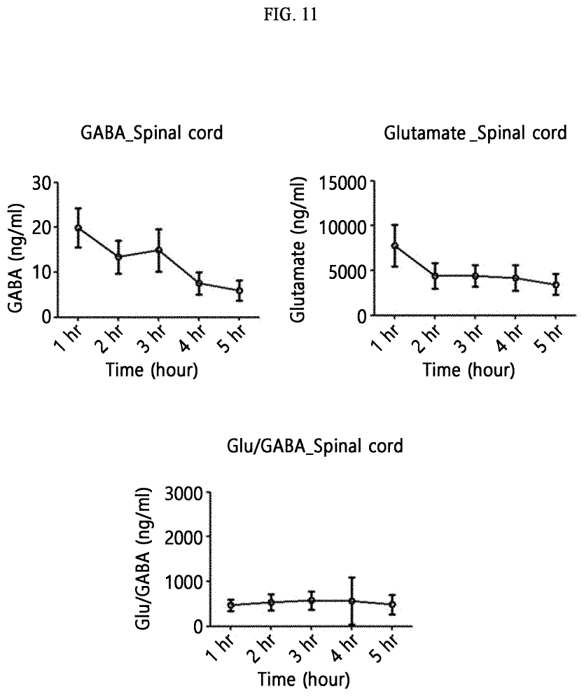

For reference, FIG. 11 shows time variation of the concentration of neurotransmitters in the spinal cord of normal rat group.

### EXAMPLE 7

### Confirmation of Possibility of Simultaneous Analysis of Neurotransmitter, Neuropeptide and Cytokine

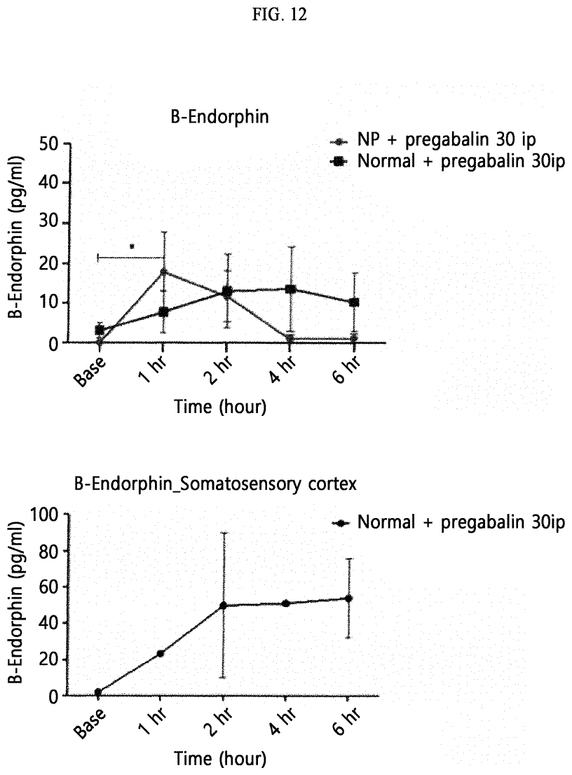

As a result of measuring concentrations of a neuropeptide and a cytokine released by the administration of 30 mg/kg of pregabalin in a neuropathic pain model and a control, concentrations of β-endorphin significantly increased and then decreased (FIG. 12).

That is, through the above result, it was confirmed that simultaneous analysis of neurotransmitters and various cytokines is possible with only one experiment.

### EXAMPLE 8

### Confirmation of Variation of Neurotransmitter According to Selective Microdialysis in Dorsal Horn of Spinal Cord and Corresponding Nerve Site

The dorsal horn in the spinal cord is a very small, it was investigated through the microdialysis method according to the method of the Example <1-4> in which part of the dorsal horn region the variation in neurotransmitter occurs.

As a result, it was confirmed that the nerve damage pathway damaged according to the Example <1-2> progresses, and the variation in neurotransmitter selectively occurs in the dorsal horn region of the spinal cord becoming a path of dorsal root ganglia (DRG).

Meanwhile, as a result of analysis to determine the number of spinal vertebra from which significant amounts of GABA and glutamate are assessed, significance for GABA and glutamate could not be obtained in a microdialysate test sample obtained from the L2 or L3 segment generally used for an NP model test.

On the other hand, as a result of confirming the DRG path of L4, which is a connective part between corresponding nerves by separating a nerve tract of an NP model, it was confirmed that the DRG path is connected to the spinal cord at the L1 segment, not the L2 segment.

Based on the result, microdialysis in the L1 segment was able to be performed by a general spinal cord microdialysis method of inserting a microdialysis probe in a cranial direction, not a caudal direction.

### EXAMPLE 9

### Confirmation of Measurable Neuropeptides and Cytokine

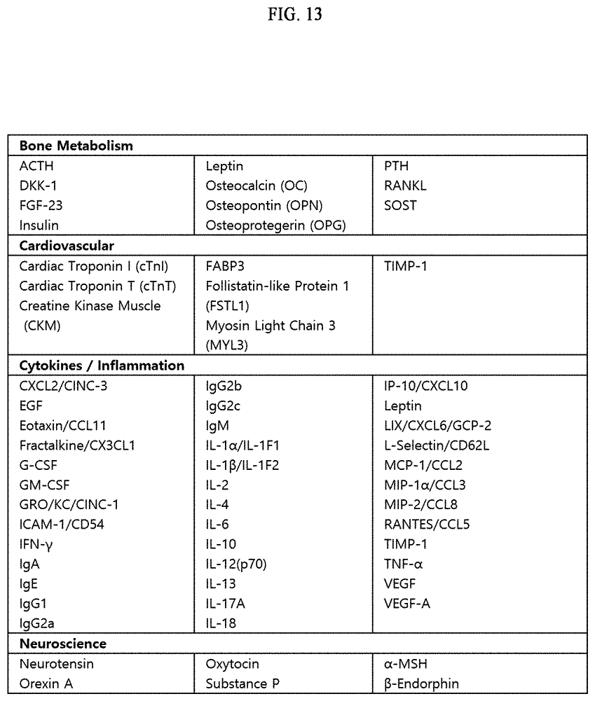

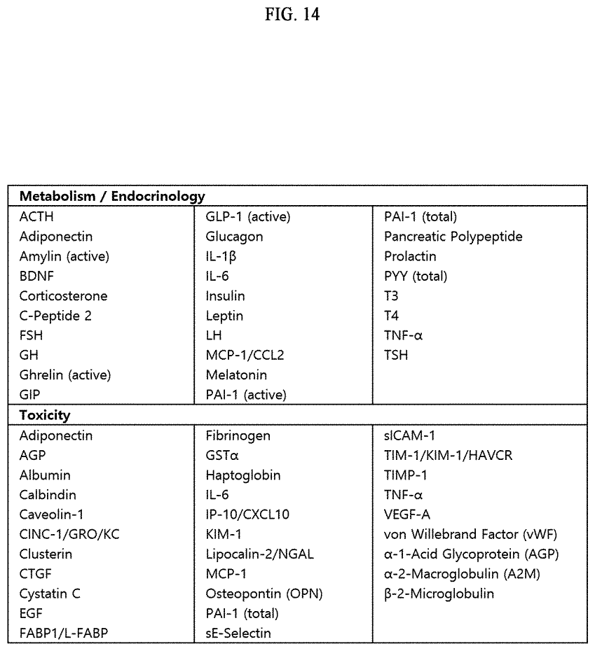

As a result of confirmation of measurable kind of neuropeptides and cytokines which can be measured by the method of <1-8> in the test sample collected in the Example <1-4>, various neuropeptides and cytokines associated with bone metabolism, cardiovascular, cytokines, inflammation, neuroscience, metabolism, endocrinology and toxicity can be confirmed (FIGS. 13 and 14).

### EXAMPLE 10

### Confirmation of Possibility of Simultaneous Analysis of Administered Drugs

To confirm whether a drug intraperitoneally administered can be analyzed at the same time as the analysis of the Example 5, the possibility of simultaneous analysis of administered drugs was analyzed according to the method of the Example <1-8>.

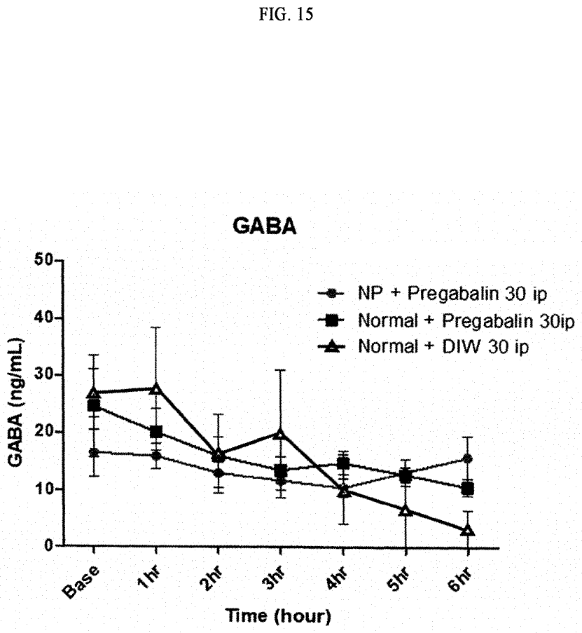

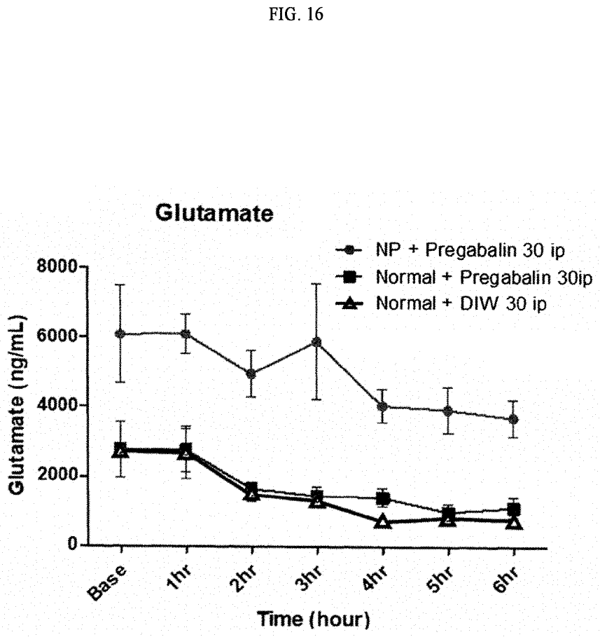

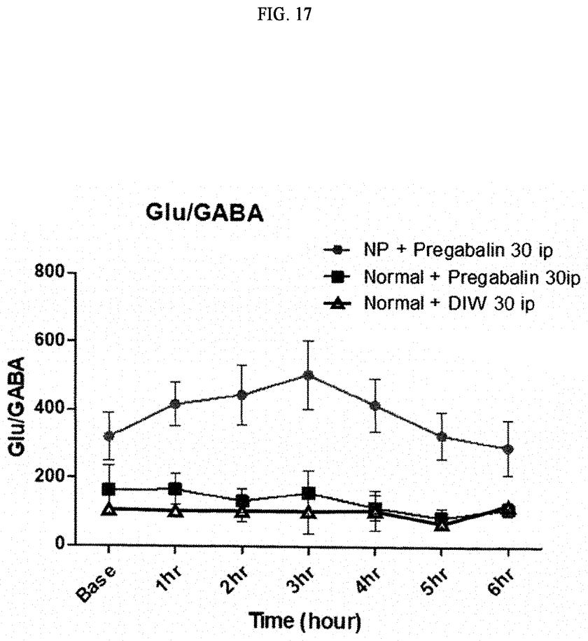

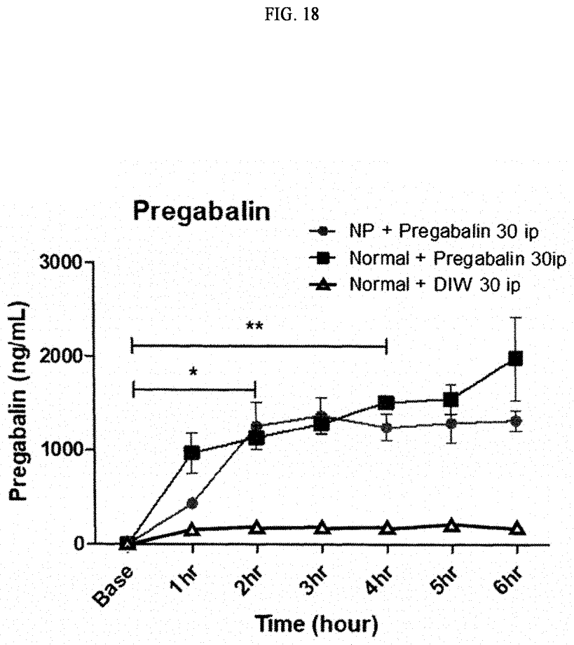

As a result, a GABA, a glutamate, a glutamate/GABA ratio and a pregabalin concentration in a microdialysate test sample were simultaneously measured (FIGS. 15 to 18), and it was confirmed that it is possible to simultaneously measure a concentration of a drug that actually acts on a target tissue by passing through a brain-blood barrier.

That is, through the above result, it was confirmed that actual PK/PD (pharmacokinetic/pharmacodynamic) models for drugs acting on the central nervous system can be established.

### EXAMPLE 11

### Confirmation of Accuracy and Precision

Accuracy and precision for each analyte were evaluated with five quality control (QC) test samples, each of corresponds to a lower limit of quantification (10 ng/mL), a low concentration (20 ng/mL), a medium concentration (100 ng/mL) and a high concentration (800 ng/mL), and inter-day accuracy and precision were measured for 5 days. The experiment was performed repeatedly five times a day in the same manner as the test sample pretreatment method to calculate coefficient of variation (CV) of GABA and glutamate, thereby obtaining intra-day precision of the calibration curve.

As a result, the coefficient of variation (CV) of GABA and glutamate were 1.9 to 4.6% and 2.0 to 2.4%, respectively, which satisfied 15% according to the Bioanalytical Method Validation Guidance, and the intra-day accuracy of GABA and glutamate were 94.9 to 104.3% and 97.6 to 106.6%, respectively, which satisfied 80 to 120%, which satisfied the Bioanalytical Method Validation Guidance (Table 3).

In addition, the inter-day precision of GABA and glutamate were 2.13.3% and 2.03.4%, respectively, which satisfied the Bioanalytical Method Validation Guidance, and the inter-day accuracies of GABA and glutamate were 96.8 to 104.9% and 99.2 to 102.4%, respectively, which satisfied the Bioanalytical Method Validation Guidance (Table 3).

TABLE 3

| | Concen- | Intra-day (n = 5) | | Inter-day (n = 5) | |
|---|---|---|---|---|---|
| | tration | Precision | Accuracy | Precision | Accuracy |
| | (ng/ml) | (CV, %) | (bias, %) | (CV, %) | (bias, %) |
| GABA | 10 | 3.3 | 102.5 | 3.3 | 104.4 |
| | 20 | 4.6 | 104.3 | 3.2 | 104.9 |
| | 100 | 1.9 | 103.2 | 2.1 | 102.3 |
| | 800 | 2.8 | 94.9 | 2.3 | 96.8 |
| Glutamate | 10 | 2.1 | 101.7 | 3.4 | 100.9 |
| | 20 | 2.0 | 106.6 | 2.5 | 102.4 |
| | 100 | 2.2 | 102.7 | 2.3 | 102.0 |
| | 800 | 2.4 | 97.6 | 2.0 | 99.2 |

That is, through the above result, it was confirmed that this analysis method for

GABA and glutamate have precision and accuracy sufficient to be applied in research using a microdialysate.

### EXAMPLE 12

### Stability

Stability was tested under various conditions for which GABA and a glutamate test sample could be exposed. Since a significant change (variation within 8%) did not occur during the storage, treatment and analysis periods of a test sample, it can be considered that GABA and glutamate are stably maintained under conditions established in this experiment (Table 4).

TABLE 4

| | | Remaining (%) | | | |
|---|---|---|---|---|---|
| | | Long term | | Micro- | Autosampler |
| | Concen- | stability | Freeze/ | dialysis | tray at |
| | tration | (−70° C. | thaw | sample at | 4° C. |
| | (ng/mL) | 1 month) | (3 cycles) | RT for 6 h | for 6 h |
| GABA | 20 | 101.8 | 103.1 | 105.3 | 105.4 |
| | 100 | 102.3 | 106.7 | 100.6 | 101.2 |
| | 800 | 97.7 | 101.6 | 91.8 | 92.1 |
| Glutamate | 20 | 104.2 | 104.1 | 101.3 | 106.5 |
| | 100 | 98.6 | 100.8 | 97.3 | 102.3 |
| | 800 | 96.0 | 101.9 | 99.8 | 101.1 |

In the present invention, a concentration of a pain indicator substance (e.g., GABA and glutamate etc.) was quantified and assessed using microdialysis from a spinal cord of a living animal which has chronic pain occurs through of a Spared nerve injury model (SNI) model, which is one of the physical allodynia models. Through this, when pain has occurred, a qualitative change of neurotransmitter in neuropathic pain was proved by confirming a mechanism of occurrence and inhibition of the pain involved in the neurotransmitter. This is a simpler and more accurate pain assessment method, compared to a conventional confirmation method.

In the past, as a pain evaluation test were mostly assessed via behavior test such as a von Frey filament test, and a test of confirming a response to a temperature such as a hot plate test and chemical pain assessment methods such as formalin were used. However, determination of pain only by a response to behavior had several problems in that an accuracy and objectiveness of an experiment are reduced. According to the present invention, using in vivo microdialysis, an intensity of pain was able to be assessed by a more accurate and objective experiment. And, by comparing the assessment of mechanical stimulation, a significant correlation in which the higher the threshold for a mechanical stimulation in an SNI model, the lower a glutamate/GABA ratio was found (*p<0.05). Therefore, it is significant that the present invention can solve the problems that still exist by providing a biological marker for objectively and quantitatively assessing pain. Furthermore, a concentration of pain-inhibiting candidate substance can be simultaneously assessed, direct assessment may be provided in pharmacokinetic and pharmacodynamic aspects.

Conventionally, there were many studies on GABA and glutamate associated with pain responses. However, it has not been quantified and assessed simultaneously in living tissue. Mainly, in order to analyze a substance, grinding spinal cord tissue, performing immunohistochemistry for a related receptor, or performing an electrophysiological test at synapses was generally performed. The present invention suggests that it is possible to perform combined assessment since amounts of neurotransmitters released in real-time from living tissue can be directly confirmed and simultaneously assessed.

In the present invention, was applied to an experiment with an SNI model to were measured GABA and glutamate, and while it is difficult to determine pain with a simple change in concentrations of two substances, respectively, it was found that a glutamate/GABA ratio can be effectively used as an pain indicator substance. Each animal had relative difference with the pain level and the release of neurotransmitters so that the relative proportions of these two substances must be combined to determine the pain patterns involved. This analysis method is useful for evaluation and development of neurotransmitters and biomarkers that are effective in studying and applying about principles of controlling pain.

The present invention confirmed that in vivo analysis of glutamate/GABA ratio in L1 dorsal horn of SM animal model can be applicable as a new biomarker, and revealed that the in vivo analysis can be applied to evaluation and comparison of drugs for neuropathic pain. In addition, it can be used as a biomarker as an evaluation method for peripheral neuropathic pain. And, it will also be used to develop drugs for treating pain.

1. A method for screening a pain-inhibiting substance, the method comprising:
(a) inserting a microdialysis probe into a L1 segment of a dorsal horn in spinal cord of a neuropathic pain animal model;
(b) collecting a first test sample from the L1 segment by microdialysis;
(c) administering a pain-inhibiting candidate substance into a body of the animal model;
(d) after the administration of the pain-inhibiting candidate substance, then collecting a second test sample from the L1 segment by microdialysis;
(e) measuring concentrations of a pain indicator substance in the first test sample and the second test sample, respectively; and
(f) comparing the concentrations of the pain indicator substance in the first test sample and the second test sample.

2. The method of claim 1, wherein the pain indicator substance is one or more selected from the group consisting of a neurotransmitter, a neuropeptide and a cytokine.

3. The method of claim 2, wherein the neurotransmitter is one or more selected from the group consisting of norepinephrine, dopamine, glutamate, γ-aminobutyric acid (GABA) and a dopamine metabolite.

4. The method of claim 2, wherein the neuropeptide is substance P or β-endorphin.

5. The method of claim 2, wherein the cytokine is one or more selected from the group consisting of MIP-1α, C5α, TNF-α, IL-1(3, IL-6, IL-15, IL-18, IFN-γ, MCP-1, CXCL1, EAA, PGEs, ATP, Nitric oxide, BDNF, c-Fos and LTs.

6. The method of claim 1, wherein the microdialysis probe is inserted into the spinal cord at an angle of 30 to 55 degrees based on the coronal.

7. The method of claim 1, wherein the microdialysis probe is inserted into the spinal cord to be located on 1.0 to 3.0 mm deep.

8. The method of claim 1, wherein the microdialysis probe is inserted so that the end of the probe faces a cranial direction of the animal model.

9. A method for screening a pain-inhibiting substance, the method comprising:
(a) inserting a microdialysis probe into a L1 segment of a dorsal horn in spinal cord of a neuropathic pain animal model;
(b) collecting a first test sample from the L1 segment by microdialysis;
(c) administering a pain-inhibiting candidate substance into a body of the animal model;
(d) after the administration of the pain-inhibiting candidate, collecting a second test sample from the L1 segment by microdialysis;
(e) measuring ratios (Glu/GABA) of concentration of a glutamate to the concentration of a γ-aminobutyric acid (GABA) concentration in the first test sample and the second test sample, respectively; and
(f) comparing the ratios of Glu/GABA concentration in the first test sample and the second test sample.

10. The method of claim 9, wherein, compared to that of the first test sample, when the Glu/GABA ratio of the second test sample is decreased, the pain-inhibiting candidate substance is selected as a pain-inhibiting substance.

11. A method for confirming that a pain-inhibiting substance acts on a L1 segment of the spinal cord dorsal horn, the method comprising:
(i) inserting a microdialysis probe into a L1 segment of a dorsal horn in spinal cord of a neuropathic pain animal model;
(ii) collecting a first test sample from the L1 segment by microdialysis for a first time;
(iii) administering a pain-inhibiting substance into a body of the animal model;
(iv) after the administration of the pain-inhibiting substance, collecting a second test sample from the L1 segment by microdialysis for a second time;
(v) measuring concentrations of a pain indicator substance in the first test sample, and concentrations of a pain indicator substance and a pain-inhibiting substance in the second test sample; and
(vi) confirming the change in concentrations of pain indicator substance in the first test sample and the second test sample, and the change in concentration of a pain-inhibiting substance in the second test sample.
